(19) United States
(12) Patent Application Publication
(10) Pub. No.: US 2024/0325580 A1
(43) Pub. Date: Oct. 3, 2024

(54) STERILIZATION MODULE AND ELECTRONIC DEVICE COMPRISING SAME

(71) Applicant: Samsung Electronics Co., Ltd., Suwon-si, Gyeonggi-do (KR)

(72) Inventors: Sunghyun CHUN, Suwon-si (KR); Jihong KIM, Suwon-si (KR); Wonhee LEE, Suwon-si (KR)

(21) Appl. No.: 18/744,257

(22) Filed: Jun. 14, 2024

Related U.S. Application Data
(63) Continuation of application No. PCT/KR2022/018197, filed on Nov. 17, 2022.

(30) Foreign Application Priority Data
Dec. 23, 2021 (KR) 10-2021-0186590

(51) Int. Cl. A61L 2/10

(52) U.S. Cl. CPC A61L 2/10; A61L 2202/11; A61L 2202/121

(57) ABSTRACT

A sterilization module according to an embodiment of the present disclosure may include: an ultraviolet unit including a light source comprising a light emitting element configured to generate ultraviolet rays; a first case including a partition wall surrounding an edge of the ultraviolet unit, the first case covering a rear surface of the ultraviolet unit; a second case covering a front surface of the ultraviolet unit, and including a first support configured to support the ultraviolet unit, a light opening configured to pass ultraviolet rays emitted from the light source, a case coupling portion configured to be coupled to the first case, a sealing portion configured to contact and press the partition wall to seal an area between the first case and the second case; and a protective window arranged on the light opening and supported by the second case.

### CROSS-REFERENCE TO RELATED APPLICATIONS

This application is a continuation of International Application No. PCT/KR2022/018197 designating the United States, filed on Nov. 17, 2022, in the Korean Intellectual Property Receiving Office and claiming priority to Korean Patent Application No. 10-2021-0186590, filed on Dec. 23, 2021, in the Korean Intellectual Property Office, the disclosures of each of which are incorporated by reference herein in their entireties.

### BACKGROUND

### Field

The disclosure relates to a sterilization module and an electronic device including the same.

### Description of Related Art

Contaminants such as mold or bacteria may be produced inside electronic devices, such as home appliances. In particular, contaminants may be more easily produced when moisture is present or formed during use of electronic devices such as air conditioners, refrigerators, or washing machines.

In order to remove such contaminants, a sterilization module for emitting ultraviolet rays to sterilize contaminants may be arranged inside the electronic device. Such sterilization modules are assembled inside electronic devices and may be required to have a waterproof function to prevent/reduce damage due to moisture.

### SUMMARY

A sterilization module according to an example embodiment may include: an ultraviolet unit including a light source configured to generate ultraviolet rays; and a first case including a partition wall at least partially surrounding an edge of the ultraviolet unit, the first case covering a rear surface of the ultraviolet unit.

The sterilization module according to an example embodiment may include: a second case covering a front surface of the ultraviolet unit, and a first support configured to support the ultraviolet unit, a light opening that passes configured to pass ultraviolet rays emitted from the light source, a case coupling portion configured to be coupled to the first case, a sealing portion configured to contact and press the partition wall to seal an area between the first case and the sealing portion.

The sterilization module according to an example embodiment may include a protective window arranged on the light opening and supported by the second case.

An electronic device according to an example embodiment may include the sterilization module according to the embodiment described above; and a module installation member comprising a support on which the sterilization module is configured to be installed.

### BRIEF DESCRIPTION OF THE DRAWINGS

The above and other aspects, features and advantages of certain embodiments of the present disclosure will be more apparent from the following detailed description, taken in conjunction with the accompanying drawings, in which:

FIG. 1 is a perspective view of an example sterilization module according to various embodiments;

FIGS. 2 and 3 are exploded perspective views of the sterilization module of FIG. 1 according to various embodiments;

FIG. 4 is a cross-sectional view of the sterilization module of FIG. 1, taken along line A-A′ according to various embodiments;

FIG. 5 is a cross-sectional view illustrating an example first case, and is an enlarged view of a portion of FIG. 4 according to various embodiments;

FIG. 6 is a perspective view of the sterilization module of FIG. 1 viewed from another angle according to various embodiments;

FIG. 7 is a diagram illustrating a state in which a wire is inserted into a wire withdrawal portion of a second case, according to various embodiments;

FIG. 8 is a cross-sectional view of the sterilization module of FIG. 6, taken along line B-B′ according to various embodiments;

FIG. 9 is a cross-sectional view illustrating an example close contact structure between a first wire withdrawal portion and a wire in the sterilization module of FIG. 6 according to various embodiments;

FIG. 10 is an exploded perspective view illustrating an example process of installing a sterilization module in an electronic device, according to various embodiments;

FIG. 11 is a perspective view for illustrating the sterilization module of FIG. 10 installed in an electronic device according to various embodiments;

FIG. 12 is a cross-sectional perspective view of the electronic device of FIG. 11 according to various embodiments;

FIG. 13 is a perspective view illustrating an example module installation member according to various embodiments;

FIG. 14 is a an exploded perspective view illustrating assembly of a module installation member with a sterilization module according to various embodiments;

FIG. 15 is a diagram illustrating an example process in which a module installation member on which a sterilization module is assembled is coupled to a frame of an electronic device according to various embodiments;

FIG. 16 is a partially exploded perspective view illustrating an example of an electronic device including a sterilization module according to various embodiments; and

FIG. 17 is a partial perspective view illustrating a state in which a sterilization module is assembled in an electronic device according to various embodiments.

### DETAILED DESCRIPTION

Hereinafter, various example embodiments of the present disclosure will be described in greater detail with reference to the accompanying drawings. Like reference numerals in the drawings indicate parts or components that perform substantially the same functions.

Terms such as “first” or “second” may be used to describe various elements, but the elements are not limited by the terms. These terms are simply used to distinguish one element from another element. For example, a first element may be referred to as a second element, and a second element may be referred to as a first element in a similar manner, without departing from the scope of the present disclosure. The term “and/or” includes any and all combinations of one or more of the associated listed items.

Terms used herein are for describing various embodiments and are not intended to limit the scope of the present disclosure. Singular forms are intended to include plural forms as well, unless the context clearly indicates otherwise. In the present disclosure, it is to be understood that the terms such as “including,” “having,” and “comprising” are intended to indicate the existence of the features, numbers, steps, actions, components, parts, or combinations thereof disclosed in the disclosure, and are not intended to preclude the possibility that one or more other features, numbers, steps, actions, components, parts, or combinations thereof may exist or may be added. Like reference numerals in the drawings indicate members that perform substantially the same functions.

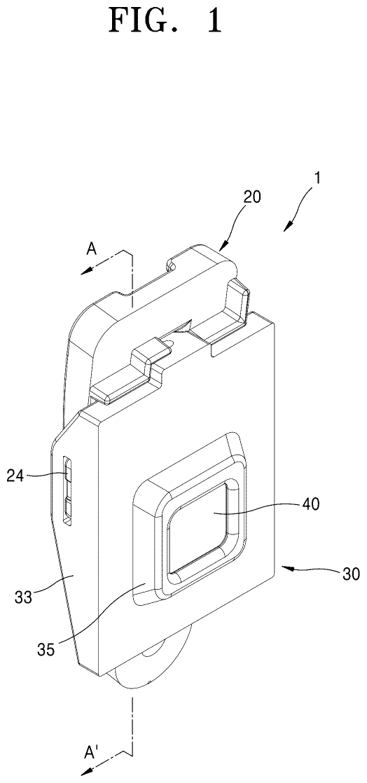

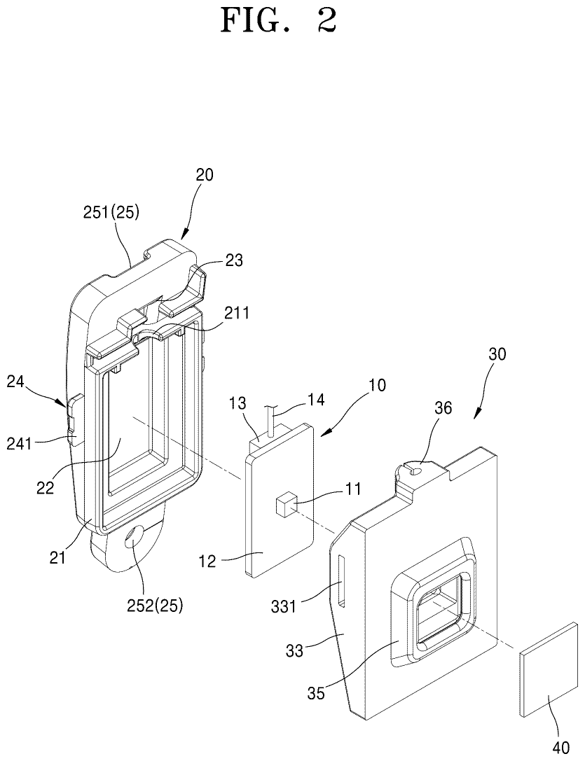

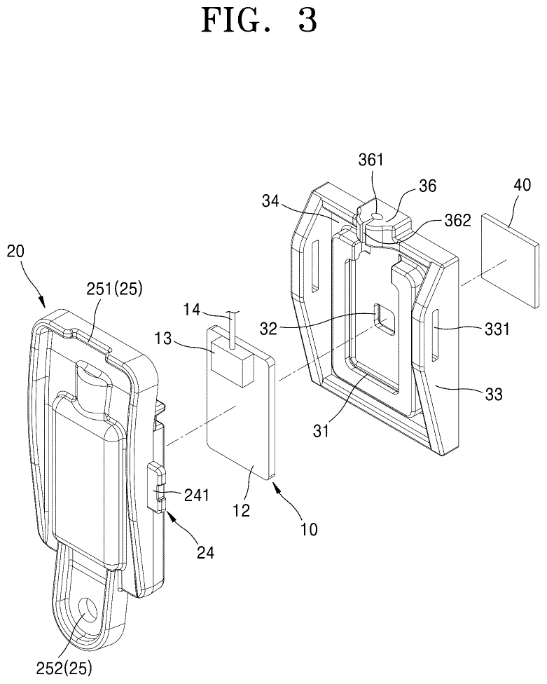

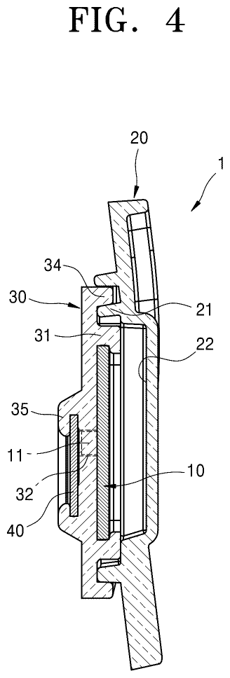

FIG. 1 is a perspective view illustrating an example sterilization module 1 according to various embodiments, and FIGS. 2 and 3 are exploded perspective views of the sterilization module 1 of FIG. 1 according to various embodiments. FIG. 4 is a cross-sectional view of the sterilization module 1 of FIG. 1, taken along line A-A′ according to various embodiments. Referring to FIGS. 1, 2, 3 and 4 (which may be referred to as FIGS. 1 to 4), the sterilization module 1 includes an ultraviolet unit (e.g., including an ultraviolet light source) 10, a first case 20, a second case 30, and a protective window 40.

The ultraviolet unit 10 may include a light source 11 configured to generate ultraviolet rays, and a substrate 12 on which the light source 11 is installed. The light source 11 may include a light-emitting element that generates ultraviolet rays. The wavelength of a ultraviolet ray region generated by the light source 11 may be a wavelength suitable for sterilization, for example, an ultraviolet C wavelength. For example, ultraviolet rays generated by the light source 11 may have a wavelength range of 100 nm to 280 nm.

The protective window 40 protects the light source 11 of the ultraviolet unit 10 and transmits ultraviolet rays generated by the light source 11. The protective window 40 may have a quadrangular shape, but is not limited thereto, and may be modified into various shapes.

The substrate 12 may have a plate shape, and the light source 11 is installed on the front surface of the substrate 12. A wire 14 may be connected to the substrate 12. The wire 14 may be connected to the substrate 12 through a connector 13. The connector 13 may be arranged on the rear surface of the substrate 12 to electrically connect the substrate 12 to the wire 14. The connector 13 may protrude from the rear surface of the substrate 12.

The first case 20 covers the rear surface of the ultraviolet unit 10 and includes a partition wall 21 surrounding an edge of the ultraviolet unit 10. The partition wall 21 surrounds an edge of the substrate 12. A groove 211 for withdrawing the wire 14 may be formed on the partition wall 21.

The first case 20 includes an insertion groove 23 provided on the outside of the partition wall 21. A portion of a wire withdrawal portion 36 of the second case 30 may be inserted into the insertion groove 23.

An accommodation groove 22 for accommodating at least a portion of the connector 13 may be provided in the first case 20. However, the accommodation groove 22 is an optional component and may be omitted depending on the shape of the ultraviolet unit 10.

The first case 20 may include a second case coupling portion 24 for coupling with the second case 30. The second case coupling portion 24 may include locking protrusions 241 provided on both sides of the first case 20. However, the shape of the second case coupling portion 24 is not limited to the foregoing and may vary depending on the shape of a case coupling portion 33 of the second case 30.

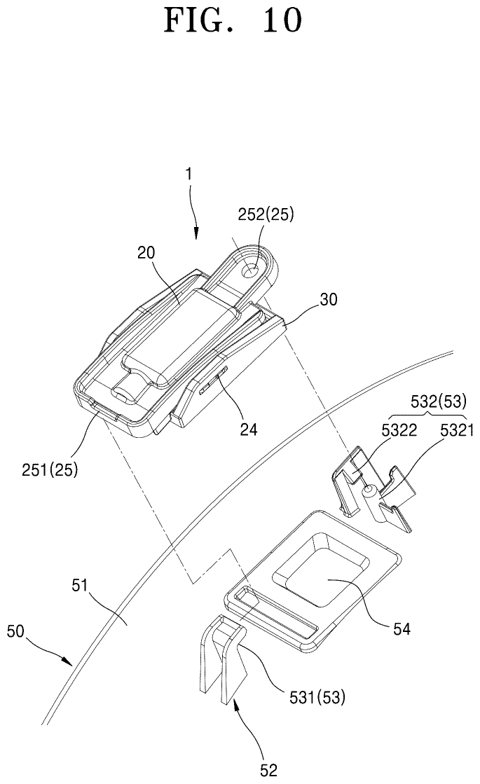

The first case 20 may include an external coupling portion 25 configured to be installed in an electronic device 50. The external coupling portion 25 may include a groove coupling portion 251 and a hole coupling portion 252. However, the configuration and structure of the external coupling portion 25 are not limited to the foregoing, and may be variously modified into any structure to be installed or coupled to another device. The external coupling portion 25 may be coupled to a module installation member 52 (see FIG. 10) of the electronic device 50 (see FIG. 10) and thus installed, which will be described below with reference to FIGS. 10 to 12.

Referring to FIGS. 2, 3 and 4, the second case 30 covers the front surface of the ultraviolet unit 10 and includes a first support 31, a light opening 32, the case coupling portion 33, and a sealing portion 34. The second case 30 further includes a second support 35. The first support 31 supports the ultraviolet unit 10, and the light opening 32 passes ultraviolet rays emitted from the light source 11 of the ultraviolet unit 10.

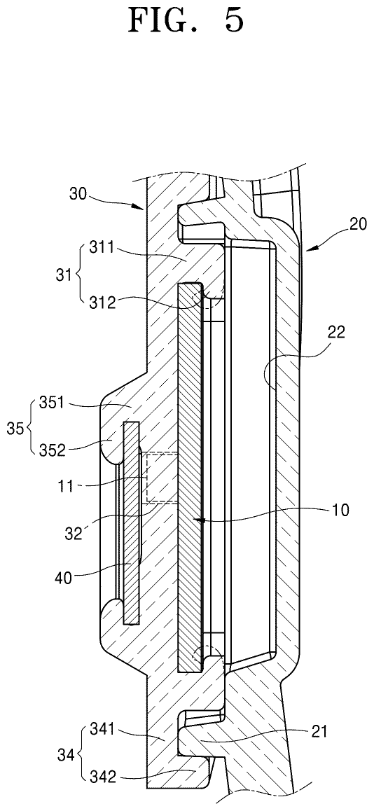

FIG. 5 is a cross-sectional view illustrating the first case 20, and is an enlarged view of a portion of FIG. 4 according to various embodiments. Referring to FIGS. 4 and 5, the first support 31 may be configured to support an edge of the ultraviolet unit 10 such that the ultraviolet unit 10 is spaced apart from the first case 20. For example, the first support 31 includes an edge support area 311 that supports an edge of the ultraviolet unit 10, and a separation prevention area 312 that prevents/blocks backward separation of the ultraviolet unit 10 supported by the edge support area 311. The edge support area 311 supports an edge of the substrate 12, and the separation prevention area 312 prevents/inhibits backward separation of the substrate 12.

The first support 31 may be elastically deformed when assembled with the ultraviolet unit 10. For example, the separation prevention area 312 of the first support 31 may be elastically deformed. Accordingly, in a process of assembling the ultraviolet unit 10 with the first case 20, the separation prevention area 312 is pressed by the substrate 12 of the ultraviolet unit 10, thus temporarily bent as illustrated by the dotted line in FIG. 5, and then restored to its original shape after the substrate 12 moves to the edge support area 311. For separating the ultraviolet unit 10 from the first support 31, the ultraviolet unit 10 may be pulled backward such that the separation prevention area 312 is temporarily bent in the opposite direction, and thus, the ultraviolet unit 10 may be easily separated from the first support 31.

As the ultraviolet unit 10 is assembled with the second case 30 through the first support 31, it is possible to determine the exact position of the ultraviolet unit 10 before the first case 20 and the second case 30 are coupled to each other, and prevent and/or reduce changes in the position of the ultraviolet unit 10 during a process of coupling the first case 20 to the second case 30.

The second support 35 may be configured to support an edge of the protective window 40. For example, the second support 35 may include a second edge support area 351 and a second separation prevention area 352. The second separation prevention area 352 may be elastically deformed. In a similar manner to the way the ultraviolet unit 10 is assembled with and separated from the first support 31, the protective window 40 may be assembled with or separated from the second support 35.

Referring again to FIGS. 1, 2 and 3, the case coupling portion 33 may be configured to couple the second case 30 to the first case 20. For example, any one of the case coupling portion 33 and the second case coupling portion 24 may include the locking protrusions 241, and the other one of the case coupling portion 33 and the second case coupling portion 24 may include locking grooves 331. For example, the second case coupling portion 24 may include the locking protrusions 241, and the case coupling portion 33 may include the locking grooves 331 into which the locking protrusions 241 may be inserted.

The case coupling portion 33 may be elastically deformed. As the second case 30 is pressed to approach the first case 20, the case coupling portion 33 is elastically deformed to be bent, and thus, the locking protrusions 241 of the second case coupling portion 24 are inserted into the locking grooves 331. As the locking protrusions 241 are inserted into the locking grooves 331, the first case 20 and the second case 30 are assembled such that the position of the second case 30 with respect to the first case 20 is fixed. For separating the second case 30 from the first case 20, an external force may be applied to the case coupling portion 33 to elastically deform the case coupling portion 33, and thus, the locking protrusions 241 may be separated from the locking grooves 331. Thus, the first case 20 and the second case 30 may be easily assembled and separated from each other.

The sealing portion 34 contacts and presses the partition wall 21 to seal an area between the first case 20 and the sealing portion 34. The sealing portion 34 may be elastically deformed. The sealing portion 34 is elastically deformed to seal the area between the first case 20 and the second case 30.

Referring to FIGS. 4 and 5, the sealing portion 34 includes a first sealing area 341 that seals an area between the first sealing area 341 and an end of the partition wall 21, and a second sealing area 342 that seals an area between the second sealing area 342 and an outer portion of the partition wall 21. The first sealing area 341 is elastically deformed to contact and press the end of the partition wall 21, and the second sealing area 342 is elastically deformed to contact and press the outer portion of the partition wall 21.

The sealing portion 34 may be arranged between the electronic device 50 and the first case 20, and may be pressed by the first case 20 and thus elastically deformed during a process of coupling the electronic device 50 to the first case 20. This will be described in greater detail below with reference to FIGS. 10, 11 and 12.

The second case 30 may include one body including an elastic material. For example, the first support 31, the light opening 32, the case coupling portion 33, the sealing portion 34, and the second support 35 may include one body. For example, the second case 30 may be one body including a rubber material. By forming the second case 30 as one body, the second case 30 may be easily manufactured and the unit cost thereof may be reduced.

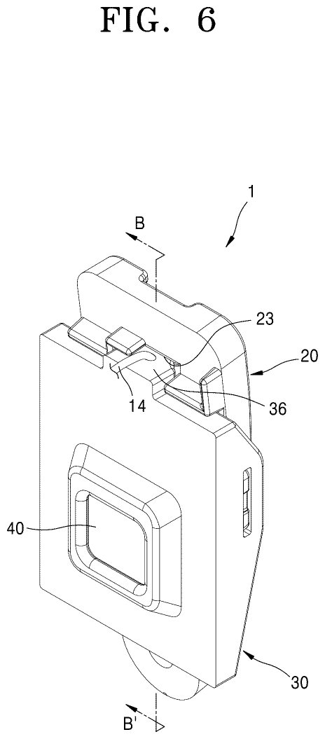

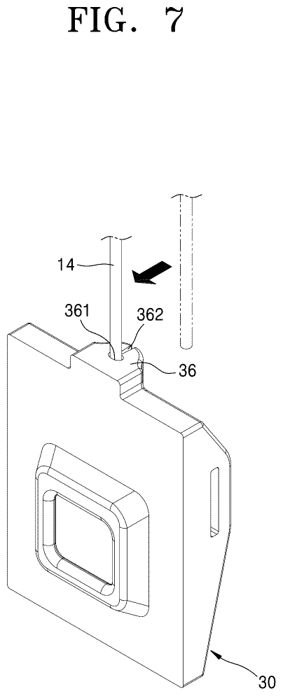

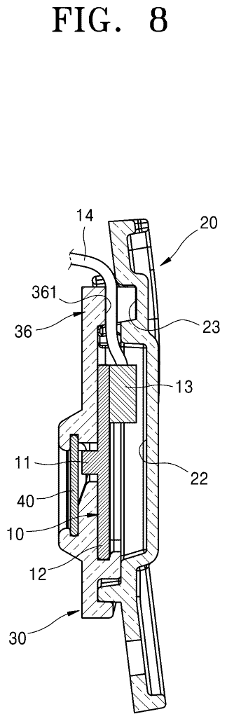

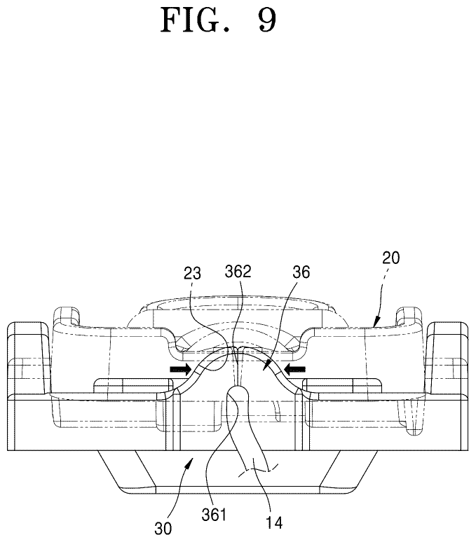

FIG. 6 is a perspective view of the sterilization module 1 of FIG. 1 viewed from another angle according to various embodiments. FIG. 7 is a perspective view illustrating a state in which the wire 14 is inserted into the wire withdrawal portion 36 of the second case 30, according to various embodiments. FIG. 8 is a cross-sectional view of the sterilization module 1 of FIG. 6, taken along line B-B′ according to various embodiments. FIG. 9 is a is a cross-sectional view illustrating an example close contact structure between the first wire withdrawal portion 36 and the wire 14 in the sterilization module 1 of FIG. 6 according to various embodiments.

Referring to FIGS. 6 and 7, the second case 30 further includes the wire withdrawal portion 36 through which the wire 14 passes. The wire withdrawal portion 36 includes a wire through hole 361 through which the wire 14 connected to the substrate 12 passes, and a cut area 362 connected to the wire through hole 361. Before the wire 14 is inserted into the wire through hole 361, the diameter of the wire through hole 361 may be less than the diameter of the wire 14.

Referring to FIG. 7, the wire 14 may be inserted into the wire through hole 361 through the cut area 362. As the wire 14 is inserted into the wire through hole 361, the diameter of the wire through hole 361 may increase to correspond to the diameter of the wire 14, and accordingly, the cut area 362 may open and increase in width.

Referring to FIGS. 6, 7 and 8, the first case 20 further includes the insertion groove 23 into which the cut area 362 of the wire withdrawal portion 36 is inserted. When the second case 30 and the first case 20 are coupled to each other, the cut area 362 of the wire withdrawal portion 36 is inserted into the insertion groove 23 of the first case 20.

Referring to FIGS. 6 and 9, in a process of inserting the cut area 362 of the wire withdrawal portion 36 into the insertion groove 23, forces are applied to the wire withdrawal portion 36 in the directions of the arrows due to the contact between the wire withdrawal portion 36 and the insertion groove 23. Accordingly, the cut area 362 may be narrowed, and the wire withdrawal portion 36 and the wire 14 may be brought into close contact with each other. Thus, it is possible to prevent moisture from penetrating between the wire withdrawal portion 36 and the wire 14.

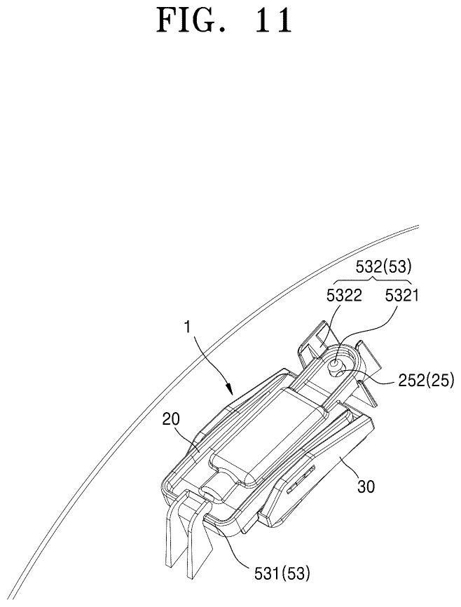

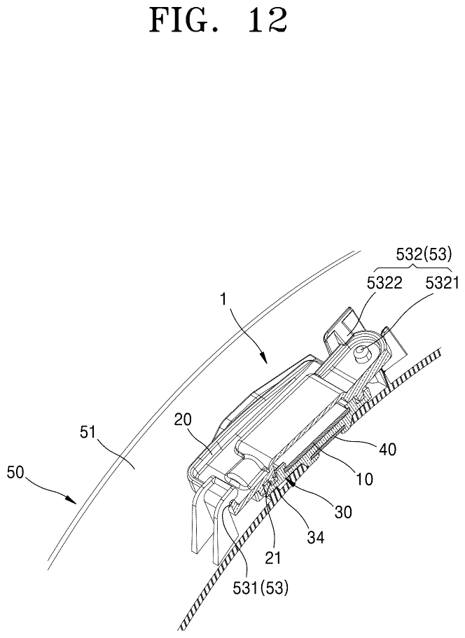

FIG. 10 is an exploded perspective view illustrating an example process of installing the sterilization module 1 in the electronic device 50, according to various embodiments, and FIG. 11 is a perspective view illustrating the sterilization module 1 of FIG. 10 installed in the electronic device 50 according to various embodiments. FIG. 12 is a cross-sectional perspective view of the electronic device 50 of FIG. 11 according to various embodiments.

Referring to FIGS. 10 and 11, the sterilization module 1 according an embodiment may be installed in the electronic device 50. The electronic device 50 may include the module installation member (e.g., a fastener) 52 on which the sterilization module 1 is installed.

The module installation member 52 may include a module coupling portion 53 configured to be assembled with the external coupling portion 25 of the first case 20. The module coupling portion 53 includes a first coupling portion 531 and a second coupling portion 532 spaced apart from each other.

The external coupling portion 25 of the first case 20 may include the groove coupling portion 251 to be supported by the first coupling portion 531, and the hole coupling portion 252 to be supported by the second coupling portion 532. The second coupling portion 532 includes the insertion protrusion 5321 to be inserted into the hole coupling portion 252, and a hook 5322 to support the hole coupling portion 252.

Hereinafter, a process of installing the sterilization module 1 in the electronic device 50 will be described in greater detail.

The sterilization module 1 may be tilted such that a portion where the groove coupling portion 251 is arranged faces downward, and then the groove coupling portion 251 is placed to be supported by the first coupling portion 531 of the module installation member 52. In a state in which the groove coupling portion 251 of the sterilization module 1 is supported by the first coupling portion 531, the insertion protrusion 5321 is placed for insertion into the hole coupling portion 252 of the sterilization module 1, and then the sterilization module 1 is pressed to approach the module installation member 52. Accordingly, the hole coupling portion 252 is moved below the hook 5322 of the module installation member 52 and remains pressed downward by the hook 5322. Thus, the sterilization module 1 is installed in the electronic device 50. Ultraviolet rays emitted from the installed sterilization module 1 pass through an opening 54 formed in a frame 51 of the electronic device 50, and then advance for irradiation.

Referring to FIG. 12, when the sterilization module 1 is installed in the electronic device 50, the second case 30 is arranged between the electronic device 50 and the first case 20. In a process of coupling the first case 20 of the sterilization module 1 to the module installation member 52, the first case 20 is pressed to approach the electronic device 50, and accordingly, the partition wall 21 presses the sealing portion 34 of the second case 30. Thus, the sealing force between the sealing portion 34 and the partition wall 21 is improved.

In an embodiment, an example in which the module installation member 52 is formed integrally with the frame 51 of the electronic device 50 is mainly described. However, the module installation member 52 is not limited to the above example, and may be a member to be assembled with the frame 51 of the electronic device 50.

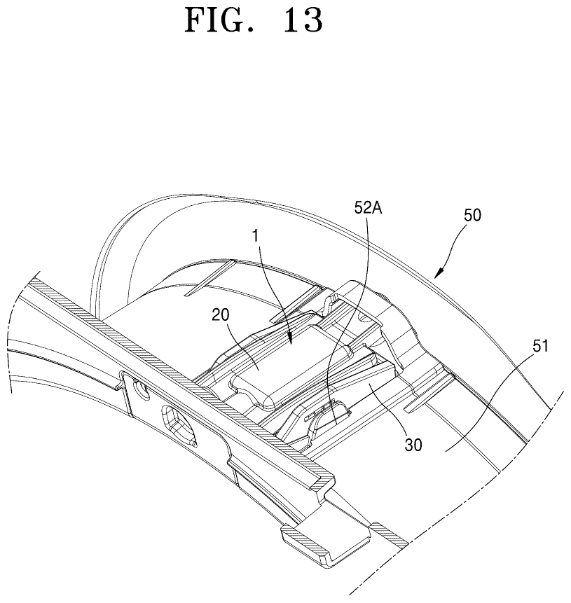

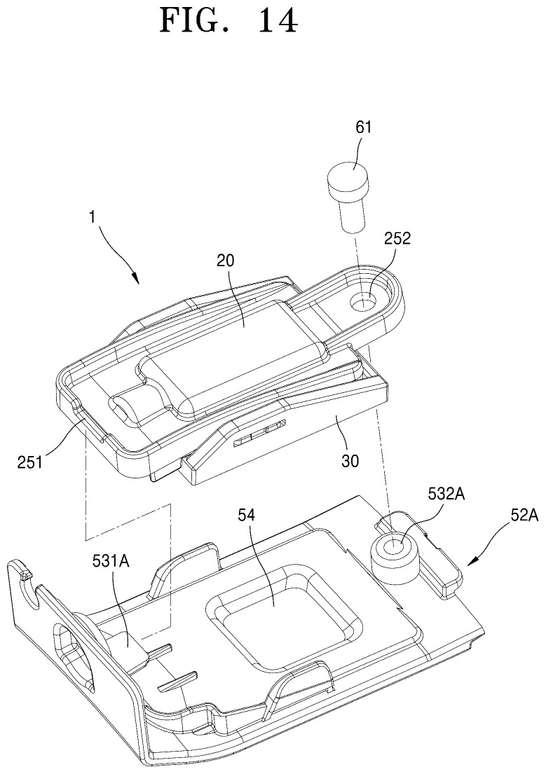

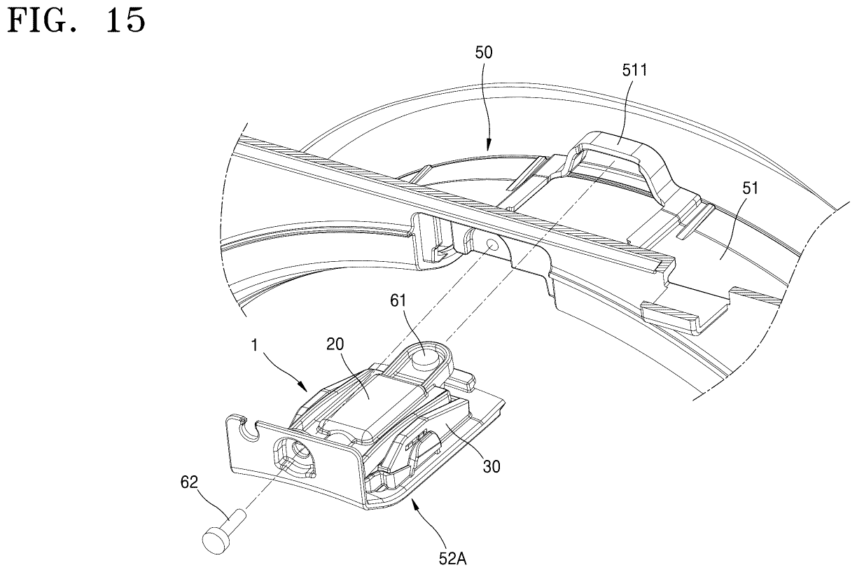

FIG. 13 is a perspective view illustrating an example module installation member 52A according to various embodiments. FIG. 14 is an exploded perspective view illustrating assembly of the module installation member 52A with the sterilization module 1 according to various embodiments, and FIG. 15 is a diagram illustrating an example process in which the module installation member 52A on which the sterilization module 1 is assembled is coupled to the frame 51 of the electronic device 50 according to various embodiments.

Referring to FIGS. 13,14 and 15, the module installation member 52A may be detachably assembled with the frame 51 of the electronic device 50. For example, one end of the module installation member 52A may be supported by the frame 51, and the other end may be fixed to the frame 51 by a fixing member 62.

Before the module installation member 52A is assembled with the frame 51, the sterilization module 1 may be coupled to the module installation member 52A. For example, the groove coupling portion 251 of the sterilization module 1 may be supported by a first coupling portion 531A of the module installation member 52A, and the hole coupling portion 252 of the sterilization module 1 may be assembled with a second coupling portion 532A of the module installation member 52A through a fixing member 61. The second coupling portion 532A and the fixing member (e.g., a fastener) 61 may be screw-coupled to each other, and in the screw coupling process, the second case 30 arranged between the module installation member 52A and the first case 20 is pressed, and thus, the sealing force between the first case 20 and the second case 30 is improved.

As such, the module installation member 52A on which the sterilization module 1 is installed is assembled with the frame 51 of the electronic device 50. Referring to FIG. 15, one end of the module installation member 52A is inserted into a support groove 511 of the frame 51, and then the other end of the module installation member 52A is fixed to the frame 51 through the fixing member (e.g., fastener) 62. Thus, the sterilization module 1 is installed in the electronic device 50.

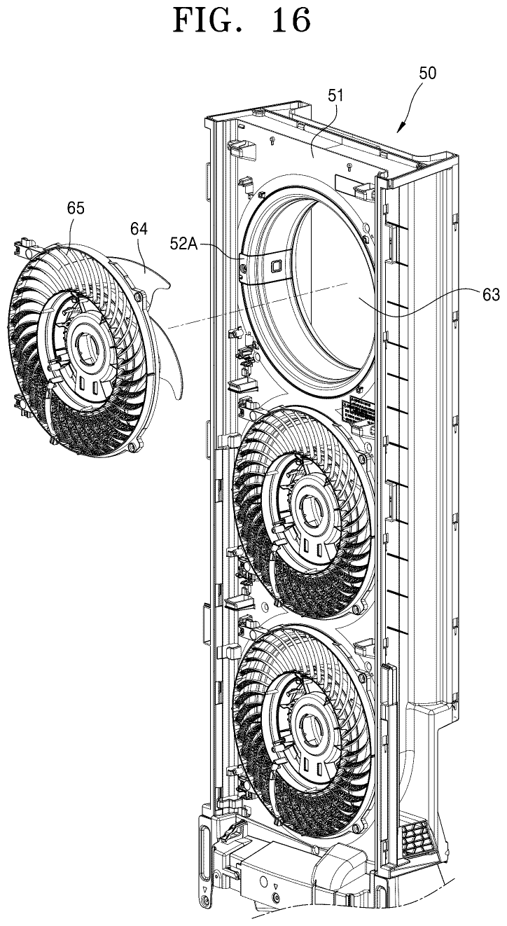

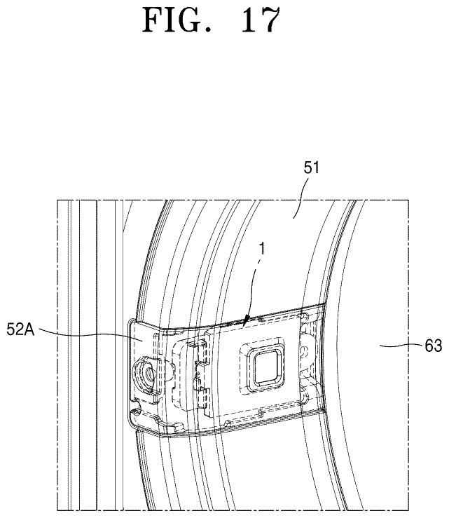

FIG. 16 is a partially exploded perspective view illustrating an example of the electronic device 50 including the sterilization module 1 according to various embodiments, and FIG. 17 is a partial perspective view illustrating a state in which the sterilization module 1 is assembled in the electronic device 50 according to various embodiments. Referring to FIGS. 16 and 17, the electronic device 50 may include a blowing fan 64 and the frame 51 supporting the blowing fan 61. A blowing grill 65 may be arranged in front of the blowing fan 64.

A fan insertion hole 63 into which the blowing fan 61 is inserted is formed in the frame 51. The sterilization module 1 according to an embodiment may be installed in the fan insertion hole. The sterilization module 1 may be supported by the module installation member 52A, and the module installation member 52A may be fixed to the frame 51. Ultraviolet rays may be emitted through the protective window 40 of the sterilization module 1.

In an embodiment, an air conditioner is described as an example of the electronic device 50 in which the sterilization module 1 is installed, but the present disclosure is not limited thereto, and the electronic device 50 may be any one of various electronic devices. For example, the electronic device 50 may be a refrigerator, a washing machine, or the like.

For the purposes of promoting understanding of the present disclosure, reference numerals are used in the various example embodiments illustrated in the drawings, and particular terms are used to describe various embodiments, however, the present disclosure is not limited by the terms and should be understood to encompass all components that would normally occur to those skilled in the art.

Particular executions described herein are merely examples and do not limit the scope of the present disclosure in any way. For the sake of brevity, related-art electronics, control systems, software and other functional aspects of the systems may not be described in detail. Furthermore, line connections or connection members between elements depicted in the drawings represent functional connections and/or physical or circuit connections by way of example, and in actual applications, they may be replaced or embodied with various suitable additional functional connections, physical connections, or circuit connections. In addition, no item or component is essential to the practice of the present disclosure unless the item or component is specifically described as being “essential” or “critical”. As used herein, the term such as “comprising”, “including” and the like are used to be understood as being an open-ended term for description.

The use of the terms “the” and similar referents in the context of describing the present disclosure (especially in the context of the following claims) are to be understood to cover both the singular and the plural. Furthermore, recitation of ranges of values herein are merely intended to serve as a shorthand method of referring individually to each separate value falling within the range, unless otherwise indicated herein, and each separate value is incorporated into the disclosure as if it were individually recited herein. The operations of the methods described herein may be performed in any suitable order unless otherwise indicated herein or otherwise clearly contradicted by context. The present disclosure is not limited to the described order of the operations. The use of any and all examples, or example language (e.g., ‘and the like’) provided herein, is intended merely to better illuminate the present disclosure and does not pose a limitation on the scope of the present disclosure unless otherwise claimed. Furthermore, various changes and modifications will be readily apparent to one of ordinary skill in the art without departing from the spirit and scope of the present disclosure.

According to an embodiment, a sterilization module having a waterproof function and a structure with excellent assemblability, and an electronic device including the sterilization module may be provided.

A sterilization module according to an example embodiment may include: an ultraviolet unit including a light source comprising a light emitting element configured to generate ultraviolet rays; a first case including a partition wall at least partially surrounding an edge of the ultraviolet unit, the first case covering a rear surface of the ultraviolet unit; a second case covering a front surface of the ultraviolet unit, and including a first support configured to support the ultraviolet unit, a light opening configured to pass ultraviolet rays emitted from the light source, a case coupling portion configured to be coupled to the first case, a sealing portion configured to contact and press the partition wall to seal an area between the first case and the second case; and a protective window arranged on the light opening and supported by the second case.

The first support may be configured to support an edge of the ultraviolet unit such that the ultraviolet unit is spaced apart from the first case.

The first support may include an edge support area to support the edge of the ultraviolet unit, and a separation prevention area configured to block rearward separation of the ultraviolet unit supported by the edge support area.

The separation prevention area may be elastically deformable.

The second case may further include a second support configured to support an edge of the protective window.

The ultraviolet unit may further include a substrate on which the light source is installed, and a wire connected to the substrate.

The ultraviolet unit may further include a connector arranged on a rear surface of the substrate and configured to connect the wire to the substrate, and the first case may include an accommodation groove configured to accommodate the connector.

The second case may further include a wire withdrawal portion having a wire through hole through which the wire passes.

The wire withdrawal portion may include a cut area connected to the wire through hole, the first case may further include an insertion groove into which the cut area of the wire withdrawal portion is configured to be inserted, and based on the second case and the first case being coupled to each other, the cut area inserted into the insertion groove may be configured to be narrowed such that the wire withdrawal portion and the wire are brought into contact with each other.

The first case may include a second case coupling portion configured to be assembled with the case coupling portion, any one of the case coupling portion and the second case coupling portion may include a locking protrusion, and another one of the case coupling portion and the second case coupling portion may include a locking groove into which the locking protrusion is insertable.

The sealing portion may include a first sealing area configured to seal an area between the first sealing area and an end of the partition wall, and a second sealing area configured to seal an area between the second sealing area and an outer portion of the partition wall.

The second case may include a body comprising an elastic material.

An electronic device according to an example embodiment may include the sterilization module according to the various embodiments described above; and a module installation member comprising a fastener on which the sterilization module is installed.

The first case of the sterilization module may include an external coupling portion configured to be installed on the electronic device, and the module installation member may include a module coupling portion configured to be coupled to the external coupling portion.

The second case may be arranged between the module installation member and the first case, and the sealing portion of the second case may be configured to be pressed by the partition wall by coupling of the external coupling portion to the module coupling portion.

The sterilization module according to various embodiments described above may prevent/reduce damage due to moisture, and may be easily assembled and separated in a manufacturing process or a repair process.

While the disclosure has been illustrated and described with reference to various example embodiments, it will be understood that the various example embodiments are intended to be illustrative, not limiting. It will be further understood by those skilled in the art that various changes in form and detail may be made without departing from the true spirit and full scope of the disclosure, including the appended claims and their equivalents. It will also be understood that any of the embodiment(s) described herein may be used in conjunction with any other embodiment(s) described herein.

What is claimed is:

1. A sterilization module comprising:
an ultraviolet unit comprising a light source comprising light emitting elements configured to generate ultraviolet rays;
a first case comprising a partition wall at least partially surrounding an edge of the ultraviolet unit, the first case covering a rear surface of the ultraviolet unit;
a second case covering a front surface of the ultraviolet unit, and comprising a first support configured to support the ultraviolet unit, a light opening configured to pass ultraviolet rays emitted from the light source, a case coupling portion configured to be coupled to the first case, and a sealing portion configured to contact and press the partition wall to seal an area between the first case and the sealing portion; and
a protective window arranged on the light opening and supported by the second case.

2. The sterilization module of claim 1, wherein the first support is configured to support an edge of the ultraviolet unit such that the ultraviolet unit is spaced apart from the first case.

3. The sterilization module of claim 1, wherein the first support comprises an edge support area configured to support the edge of the ultraviolet unit, and a separation prevention area configured to block rearward separation of the ultraviolet unit supported by the edge support area.

4. The sterilization module of claim 3, wherein the separation prevention area is elastically deformable.

5. The sterilization module of claim 1, wherein the second case further comprises a second support configured to support an edge of the protective window.

6. The sterilization module of claim 1, wherein the ultraviolet unit further comprises a substrate on which the light source is installed, and a wire connected to the substrate.

7. The sterilization module of claim 6, wherein the ultraviolet unit further comprises a connector arranged on a rear surface of the substrate and configured to connect the wire to the substrate, and
the first case comprises an accommodation groove configured to accommodate the connector.

8. The sterilization module of claim 6, wherein the second case further comprises a wire withdrawal portion having a wire through hole through which the wire is configured to pass.

9. The sterilization module of claim 8, wherein the wire withdrawal portion comprises a cut area connected to the wire through hole,
the first case further comprises an insertion groove into which the cut area of the wire withdrawal portion is configured to be inserted, and
based on the second case and the first case being coupled to each other, the cut area inserted into the insertion groove is configured to be narrowed such that the wire withdrawal portion and the wire are brought into contact with each other.

10. The sterilization module of claim 1, wherein the first case comprises a second case coupling portion configured to be assembled with the case coupling portion,
any one of the case coupling portion and the second case coupling portion comprises a locking protrusion, and
another one of the case coupling portion and the second case coupling portion comprises a locking groove into which the locking protrusion is insertable.

11. The sterilization module of claim 1, wherein the sealing portion comprises a first sealing area configured to seal an area between the first sealing area and an end of the partition wall, and a second sealing area configured to seal an area between the second sealing area and an outer portion of the partition wall.

12. The sterilization module of claim 1, wherein the second case includes one body comprising an elastic material.

13. An electronic device comprising:
a sterilization module of claim 1; and
a module installation member comprising a fastener on which the sterilization module is configured to be installed.

14. The electronic device of claim 13, wherein the first case of the sterilization module comprises an external coupling portion configured to be installed on the electronic device, and
the module installation member comprises a module coupling portion configured to be coupled to the external coupling portion.

15. The electronic device of claim 13, wherein the second case is arranged between the module installation member and the first case, and
the sealing portion of the second case is configured to be pressed by the partition wall by coupling of the external coupling portion to the module coupling portion.
